(19) United States
(12) Patent Application Publication
(10) Pub. No.: US 2015/0037848 A1
(43) Pub. Date: Feb. 5, 2015

(54) METHOD FOR PRODUCING SACCHARIDE

(71) Applicant: KAO CORPORATION, Tokyo (JP)

(72) Inventor: Toshihiro Yano, Wakayama-shi (JP)

(73) Assignee: KAO CORPORATION, Tokyo (JP)

(21) Appl. No.: 14/384,922

(22) PCT Filed: Feb. 21, 2013
(86) PCT No.: PCT/JP2013/054363

§ 371 Date: Sep. 12, 2014

(30) Foreign Application Priority Data
Mar. 15, 2012 (JP) 2012-059223
Feb. 1, 2013 (JP) 2013-018531

(51) Int. Cl. C12P 19/14

(52) U.S. Cl. CPC C12P 19/14
USPC 435/99

(57) ABSTRACT

The invention provides a method for producing saccharide, the method including the following steps (1) and (2). In step (1), a cellulose raw material is heated in the presence of one or more additives selected from the group consisting of a nonionic surfactant and polyethylene glycol; and water, at a pH lower than 7, to thereby prepare a heat treatment product. In step (2), the treatment product prepared in step (1) to is subjected to a saccharification treatment by use of an enzyme.

### FIELD OF THE INVENTION

The present invention relates to a method for producing saccharide.

### BACKGROUND OF THE INVENTION

In recent years, from the viewpoint of, for example, solving environmental problems, attempts have been made to produce a saccharide from a cellulose-containing biomass material and to convert the saccharide to ethanol or lactic acid through fermentation or a similar technique. In a method for producing saccharide by treating a cellulose-containing biomass material with an enzyme such as cellulase, and saccharifying the cellulose, it is useful to carry out decrystallization of the crystal structure of the cellulose, or removal of lignin from the biomass material, in a preliminary treatment step. As has been disclosed, in such a preliminary treatment step, for example, cellulose is decrystallized by use of a cellulose solvent such as lithium chloride/dimethylacetamide (see, for example, Patent Document 1).

There has been also disclosed a process for saccarification of cellulose contained in a biomass material by the mediation of an enzyme. As has been disclosed, a preliminary treatment step of the process includes a treatment with hot water and hydrogen peroxide, to thereby decompose lignin contained in the biomass material for removal (Patent Documents 2 and 3).

There have been known some cellulose saccharification processes including a process in which cellulose is saccharified by use of a specific cellulase (Patent Document 4); and a process in which a lignocellulose material is saccharified by use of a cellulase in the presence of a specific nonionic surfactant (Patent Document 5).

### PRIOR ART DOCUMENT

### Patent Document

Patent Document 1: Japanese Patent Application Laid-Open (kokai) No. 2006-223152
Patent Document 2: Japanese Patent Application Laid-Open (kokai) No. 2007-74992
Patent Document 3: Japanese Patent Application Laid-Open (kokai) No. 2007-74993
Patent Document 4: Japanese Patent Application Laid-Open (kokai) No. 2003-135052

### Patent Document 5: WO2005/067531

### SUMMARY OF THE INVENTION

### Problems to be Solved by the Invention

However, the processes described in Patent Documents 1 to 5 are unsatisfactory in terms of saccharification efficiency and productivity. Thus, the saccharification efficiency has desired to be improved.

### Means for Solving the Problems

The present inventor has found that the aforementioned problems can be solved by heating a cellulose raw material under specific conditions and then subjecting the heat treatment product to saccharification with an enzyme.

Accordingly, the present invention provides a method for producing saccharide, including the following steps (1) and (2).

step (1): a step of heating a cellulose raw material in the presence of one or more additives selected from the group consisting of a nonionic surfactant and polyethylene glycol; and water, at a pH lower than 7, to thereby prepare a heat treatment product

step (2): a step of subjecting the treatment product prepared in step (1) to a saccharification treatment by use of an enzyme.

### Effects of the Invention

According to the method for producing saccharide of the present invention, cellulose is saccharified with improved efficiency through the enzymatic treatment, and a saccharide can be effectively produced from the cellulose raw material.

### DETAILED DESCRIPTION OF THE INVENTION

### Method for Producing Saccharide

The method for producing saccharide of the present invention includes the aforementioned steps (1) and (2). The reason for remarkable enhancement in saccharification efficiency has not been completely elucidated. One conceivable reason is that, when a cellulose raw material is subjected to the heating treatment of step (1) in the co-presence of water and a specific additive, removal of hemicellulose and lignin, lowering of the molecular weight of cellulose, and increase in specific surface area of cellulose are promoted, whereby saccharification efficiency can be enhanced.

### <Step (1)>

In step (1), a cellulose raw material is heated in the presence of one or more additives selected from the group consisting of a nonionic surfactant and polyethylene glycol; and water, at a pH lower than 7, to thereby prepare a heat treatment product.

### (Cellulose Raw Material)

No particular limitation is imposed on the type of the cellulose raw material. Examples of the cellulose raw material include wood materials obtained from conifers such as larch and Japanese cypress and hardwoods such as oil palm and sawtooth oak; pulp such as wood pulp produced from wood materials, and cotton linter pulp obtained from fiber surrounding cotton seeds; paper such as newspaper, corrugated cardboard, magazine, and wood-free paper; stems, leaves, and fruit bunches of plants, such as bagasse (residue of squeezed sugarcane), palm empty fruit bunch (EFB), rice straw, and corn stems; and shells of plants, such as chaff, palm shells, and coconut shells.

From the viewpoints of improvement in saccharification efficiency, easy availability, and raw material cost, the aforementioned raw material is preferably wood materials stems, leaves, and fruit bunches of plants, more preferably bagasse, EFB, and oil palm (stem), still more preferably bagasse.

The holocellulose content of the cellulose raw material employed in the present invention is preferably 20 mass % or more, more preferably 40 mass % or more, even more preferably 45 mass % or more, still more preferably 50 mass % or more. The holocellulose content may be determined through the method described in the Examples.

### (Preliminary Treatment of Cellulose Raw Material)

Before carrying out step (1), the cellulose raw material employed in the present invention is preferably subjected to a preliminary treatment such as cutting treatment or rough milling treatment, depending on the size or shape of the raw material, for improving handling property and saccharification efficiency.

### [Cutting Treatment]

Before carrying out step (1), the cellulose raw material employed in the present invention is preferably subjected, in advance, to cutting treatment, depending on the size or shape of the raw material.

Examples of the means for cutting the cellulose raw material include one or more cutting machines selected from among a shredder, a slitter cutter, and a rotary cutter.

When a sheet-form cellulose raw material is used, a shredder or a slitter cutter is preferably employed as a cutting machine. From the viewpoint of productivity, a slitter cutter is more preferably employed.

When a slitter cutter is employed, a sheet-form raw material is cut along the longitudinal direction thereof by means of a roller cutter, to thereby provide long strips, and the long strips are cut into short pieces along the transverse direction by means of fixed blades and rotary blades, to thereby readily provide dice-form cellulose raw material pieces. As a slitter cutter, a sheet pelletizer (available from HORAI Co., Ltd.), a Super Cutter (available from Ogino Seiki Co., Ltd.), or the like is preferably employed. By means of these machines, a sheet-form cellulose raw material can be cut into square pieces having dimensions of about 1 to about 20 mm×about 1 to about 20 mm) pieces.

In the case where a wood material such as thinning waste, pruned-off material, or building waste, or a non-sheet cellulose raw material is cut, a rotary cutter is preferably employed. A rotary cutter includes rotating blades and a screen. By the action of the rotating blades, cut pieces of the cellulose raw material having a size smaller than the opening size of the screen can be readily provided. If required, a fixed blade may be added thereto, and the raw material can be cut by means of the rotating blades and the fixed blades.

When a rotary cutter is employed, the size of the cut product may be regulated by modifying the opening size of the screen. The opening size of the screen is preferably 1 to 70 mm, more preferably 2 to 50 mm, still more preferably 3 to 40 mm. When the screen has an opening size of 1 mm or more, a cut product having an appropriate bulk density can be produced, and the handling property thereof is enhanced. When the screen has an opening size of 70 mm or less, the product has a piece size suitable as a raw material to be subjected to milling in the below-mentioned rough milling treatment, and the load required for milling can be reduced.

The square piece size of the cellulose raw material after cutting treatment is preferably 1 to 70 mm×1 to 70 mm, more preferably 2 to 50 mm×2 to 50 mm. When each piece has a square size of 1 to 70 mm×1 to 70 mm, a post drying treatment can efficiently and readily performed, and the load required for milling in the below-mentioned rough milling treatment can be reduced.

### [Rough Milling Treatment]

Before carrying out step (1), the cellulose raw material employed is preferably subjected, in advance, to rough milling treatment. Also, the cellulose raw material which has been subjected to cutting treatment may be further subjected to rough milling treatment.

The rough milling treatment may be carried out by means of a known mill. No particular limitation is imposed on the mill employed, so long as it can reduce the particle size of the cellulose raw material.

Specific examples of the mill include roll mills such as a high-pressure compression roll mill and a roll-rotating mill; vertical roller mills such as a ring roller mill, a roller race mill, and a ball race mill; container-driving medium mills such as a rolling ball mill, a vibration ball mill, a vibration rod mill, a vibration tube mill, a planetary ball mill, and a centrifugal fluidization mill; medium-stirring mills such as a tower pulverizer, an agitation tank mill, a flowing tank mill, and an annular mill; compaction shearing mills such as a high-speed centrifugal roller mill and an angmill; a mortar; a stone mill; a mass colloider; a fret mill; an edge-runner mill; a knife mill; a pin mill; and a cutter mill. Of these, a container-driving medium mill or a medium-stirring mill is preferred, a container-driving medium mill is more preferred, a vibration mill selected from among a vibration ball mill, a vibration rod mill, and a vibration tube mill is much more preferred, and a vibration rod mill is much more preferred, from the viewpoints of the milling efficiency of the cellulose raw material and productivity.

The milling treatment may be carried out in a batch or continuous process.

No particular limitation is imposed on the material of the apparatus and/or medium employed for rough milling. Examples of the material include iron, stainless steel, alumina, zirconia, silicon carbide, silicon nitride, and glass. In order to effectively reduce the crystallinity of cellulose, iron, stainless steel, zirconia, silicon carbide, or silicon nitride is preferably employed. Also, from the viewpoint of industrial application, iron or stainless steel is preferably employed.

When a vibration mill is employed, and rods are employed as media therefor, the outer diameter of the rods is preferably 0.1 to 100 mm, more preferably 0.5 to 50 mm, for improvement of the milling efficiency of the cellulose raw material. When the size of the rods falls within the above range, the particle size of the cellulose raw material can be effectively reduced, and contamination of the cellulose, which would otherwise be caused by inclusion of fragments of the rods thereinto, can be suppressed.

The percent filling of rods in the vibration mill, which may vary with the type of the mill employed, is preferably 10 to 97%, more preferably 15 to 95%, even more preferably 30 to 80%. When the percent filling falls within the above range, the frequency of contact between the cellulose raw material and the rods increases, and the milling efficiency of the cellulose raw material can be improved without inhibiting the motion of the rods. As used herein, the “percent filling” refers to the ratio of the apparent volume of the rods to the volume of the stirring unit of the vibration mill.

No particular limitation is imposed on the temperature during the rough milling treatment. However, from the viewpoints of operation cost and suppression of degradation of the cellulose raw material, the treatment temperature is preferably −100 to 200° C., more preferably 0 to 150° C., even more preferably 5 to 100° C.

The milling time may be appropriately adjusted so that the particle size of the cellulose raw material is reduced through the rough milling treatment. The milling time, which may vary with, for example, the type of the mill employed or the amount of energy employed, is generally 1 to 30 minutes. From the viewpoints of reduction of the particle size of the cellulose raw material and energy cost, the milling time is preferably 2 to 15 minutes, more preferably 2 to 10 minutes.

### (Additives)

In step (1), one or more additives selected from the group consisting of a nonionic surfactant and polyethylene glycol are used, from the viewpoint of enhancing saccharification efficiency.

### [Nonionic Surfactant]

The nonionic surfactant serving as an additive in step (1) is preferably a nonionic surfactant having a polyoxyethylene moiety or a polyhydric alcohol moiety, from the viewpoint of enhancement in saccharification efficiency, more preferably a nonionic surfactant having a polyoxyethylene moiety. The average addition amount by mole of polyoxyethylene groups in the nonionic surfactant is preferably 5 to 200 from the viewpoint of enhancement in saccharification efficiency, more preferably 10 to 150, even more preferably 12 to 120. Also from the viewpoint of enhancement in saccharification efficiency, the nonionic surfactant preferably has high hydrophilicity. More specifically, the nonionic surfactant preferably has a hydrophil-lipophyl balance (HLB value) calculated through the Griffin method of 3 to 20, more preferably 5 to 20, even more preferably 8 to 20, yet more preferably 10 to 20, yet more preferably 12 to 20, yet more preferably 16 to 20.

Examples of preferred nonionic surfactants include polyoxyethylene alkyl ethers, polyoxyethylene sorbitan fatty acid esters, polyoxyethylene sorbitol fatty acid esters, polyoxyethylene fatty acid esters, polyoxyethylene hydrogenated castor oils, and sorbitan fatty acid esters. Of these, from the viewpoint of enhancement in saccharification efficiency, one or more nonionic surfactants selected from the group consisting of polyoxyethylene alkyl ethers, polyoxyethylene sorbitan fatty acid esters, polyoxyethylene sorbitol fatty acid esters, and polyoxyethylene fatty acid esters are preferred, with one or more nonionic surfactants selected from the group consisting of polyoxyethylene sorbitan fatty acid esters and polyoxyethylene fatty acid esters being more preferred. Particularly, polyoxyethylene sorbitan fatty acid esters are even more preferred. More specifically, one or more nonionic surfactants selected from the group consisting of polyoxyethylene sorbitan laurate, polyoxyethylene sorbitan stearate, polyoxyethylene lauryl ether, polyethylene glycol monolaurate, and polyethylene glycol monostearate are still more preferred, with one or more nonionic surfactants selected from the group consisting of polyoxyethylene sorbitan laurate and polyoxyethylene sorbitan stearate being further more preferred.

### [Polyethylene Glycol]

The polyethylene glycol serving as an additive in step (1) preferably has a weight average molecular weight 200 to 20,000, from the viewpoint of enhancement in saccharification efficiency, more preferably 300 to 18,000, even more preferably 400 to 15,000, still more preferably 1,000 to 10,000, yet more preferably 1,500 to 6,000. Meanwhile, the weight average molecular weight of polyethylene glycol is determined through, for example, gel permeation chromatography (GPC).

From the viewpoint of enhancement in saccharification efficiency, the total amount of the additives used in step (1) is preferably 0.1 to 100 mass %, with respect to the weight of the dry cellulose raw material, more preferably 0.2 to 80 mass %, even more preferably 0.2 to 50 mass %, still more preferably 1 to 20 mass %.

No particular limitation is imposed on the method of adding the additive to the cellulose raw material, and the additive may be added in a single batch or in a divided manner. In order to attain uniform dispersion of the additive, in one preferred mode, an additive or additives are added to a mixture of water and a cellulose raw material, and the mixture is stirred. In another preferred mode, an additive or additives are added to a mixture of water and a cellulose raw material, while the resultant mixture is continuously stirred. In an alternative mode, an additive or additives are added to water, and then a cellulose raw material is added thereto.

Addition of the additive may be carried out in an apparatus employed for the below-described heating treatment, or in a separate stirring/mixing apparatus. No particular limitation is imposed on the aforementioned stirring/mixing apparatus, so long as the apparatus can disperse the additive in the cellulose raw material. Examples of the apparatus include a ribbon type mixer, a paddle type mixer, a conical planetary screw type mixer, and a kneader employed for kneading of powder, high-viscosity material, resin, etc. Among these apparatuses, a horizontal axis paddle type mixer is more preferably employed. Specifically, there is still more preferably employed a Lodige mixer (product of Chuoh Kikoh; the mixer using a characteristic plow-like shovel and capable of being provided with a chopper blade), which is a horizontal axis paddle type mixer having a chopper blade, or a ploughshare mixer (product of Pacific Machinery & Engineering Co., Ltd.; a mixer having the following two functions: floating diffusion mixing by a shovel blade having a unique shape, and high-speed shearing dispersion by a multi-stage chopper blade).

### (Heating Treatment)

In step (1), the cellulose raw material is heated in the presence of the aforementioned additives and water, at a pH lower than 7. In one preferred mode thereof, a roughly milled product of the cellulose raw material is dispersed in water with the additives, to thereby prepare an aqueous slurry, and the slurry is subjected to the heating treatment.

In order to improve flowability, the slurry preferably has a cellulose raw material content of 1 to 500 g/L, more preferably 5 to 400 g/L, even more preferably 8 to 300 g/L.

No particular limitation is imposed on the procedure of the heating treatment, and a known method may be applied. A reactor of a batch type, a continuous type, or the like may be employed. No particular limitation is imposed on the type of the reactor, so long as the reactor can heat the slurry. Since heating treatment is performed at a pH lower than 7 in step (1), a reactor which can be employed under acidic conditions is preferably employed. In the case where the heating treatment of step (1) is performed in a pressurized atmosphere, a reactor equipped with a pressure regulating mechanism such as a back pressure regulating valve is preferably employed.

The heating treatment of step (1) is performed at a pH lower than 7, for removing miscellaneous matter such as hemicellulose and lignin, for lowering the molecular weight of cellulose, or for increasing the specific surface area of cellulose, and for other reasons. From the same viewpoints, the pH is preferably 4 or lower, more preferably 0.1 to 3.0, even more preferably 0.5 to 2.7, still more preferably 1.0 to 2.5. Examples of the pH regulating agent for adjusting the pH include inorganic acids such as sulfuric acid, hydrochloric acid, nitric acid, and phosphoric acid, and organic acids such as formic acid, acetic acid, and citric acid. Among these acids, hydrochloric acid and sulfuric acid are preferred, for removing miscellaneous matters such as hemicellulose and lignin, for lowering the molecular weight of cellulose, or for increasing the specific surface area of cellulose, with sulfuric acid being more preferred.

The temperature at which the heating treatment is performed is preferably 100 to 300° C., more preferably 120 to 250° C., even more preferably 140 to 230° C., for the purposes of removing miscellaneous matters such as hemicellulose, enhancing saccharification efficiency, and preventing excessive degradation of saccharide. The rate of temperature elevation and cooling, and the retention time at the peak heating temperature may be appropriately controlled.

The pressure during the heating treatment of step (1) is preferably a pressure equal to or higher than a saturated steam pressure, for removing miscellaneous matter such as hemicellulose and lignin, for lowering the molecular weight of cellulose, or for increasing the specific surface area of cellulose, and for other reasons. The pressure is more preferably 0.1 to 10 MPa, even more preferably 0.2 to 8 MPa, still more preferably 0.3 to 6 MPa, particularly preferably 0.5 to 6 MPa. Examples of the pressurizing gas include an inert gas, steam, nitrogen gas, and helium gas. Alternatively, instead of using the pressurizing gas, pressurization may be performed through regulating the pressure by means of a back pressure regulating valve of the reactor.

In step (1), the treatment time (average retention time) varies depending on the treatment conditions and the type of cellulose raw material. However, from the viewpoint of enhancement in saccharification efficiency and productivity, the treatment time from the time at which the slurry has been heated to a target temperature is preferably 0.5 to 60 minutes, more preferably 1 to 30 minutes, even more preferably 1 to 20 minutes, in the case where the reaction is performed in a continuous manner.

From the viewpoint of enhancement in saccharification efficiency, in a preferred mode, the slurry after the heating treatment is cooled to preferably 100° C. or lower, more preferably 60° C. or lower. Through performing step (1) in the aforementioned manner, a treatment product is prepared. The treatment product generally assumes the form of aqueous slurry.

### <Neutralization Step>

In the method for producing saccharide of the present invention, from the viewpoint of enhancement in saccharification efficiency in the below-described step (2), the treatment product prepared in step (1) is preferably subjected to a step of neutralizing the product with a base (hereinafter may be referred to as a “neutralization step”) before use in step (2).

Examples of the base used in the neutralization step include inorganic bases such as sodium hydroxide, potassium hydroxide, and calcium hydroxide; and organic bases such as ammonia and organic amines. Of these, sodium hydroxide, potassium hydroxide, and calcium hydroxide are preferred, from the viewpoint of enhancement in productivity, saccharification efficiency, and yield of saccharide, with sodium hydroxide or calcium hydroxide being more preferred. No particular limitation is imposed on the neutralization method using a base, but an aqueous solution of the base is preferably used for neutralization.

The resultant product which has been subjected to the neutralization step preferably has a pH of 4.5 to 7.5.

### <Step (2)>

In step (2), the treatment product prepared through step (1) is subjected to saccharification treatment by use of an enzyme.

From the treatment product prepared through step (1), miscellaneous matters such as hemicellulose and lignin have been removed. The cellulose contained in the treatment product has low molecular weight and increased specific surface area. Therefore, treatment of the above product with an enzyme can effectively produce a monosaccharide such as glucose or xylose, and a mixture of, for example, oligosaccharides such as cellobiose, cellotriose, xylobiose, and xylotriose. Preferably, the saccharification treatment is carried out to form monosaccharides, in consideration of the case where, for example, the thus-saccharified product is subjected to ethanol fermentation or lactic fermentation.

For improvement of saccharification efficiency, the enzyme employed in step (2) may be, for example, cellulase or hemicellulase.

As used herein, the term “cellulose” refers to enzymes which hydrolyze a glycoside bond of β-1,4-glucan of cellulose and collectively refers to enzymes including endoglucanase, exoglucanase, cellobiohydrolase, and β-glucosidase. Examples of the cellulase employed in the present invention include commercially available products of cellulase, and those derived from animals, plants, and microorganisms.

Specific examples of the cellulase include cellulase products derived from Trichoderma reesei, such as Celluclast 1.5L (trade name, product of Novozymes); cellulase derived from a Bacillus sp. KSM-N145 strain (FERM P-19727); cellulase derived from strains of Bacillus sp. KSM-N252 (FERM P-17474), Bacillus sp. KSM-N115 (FERM P-19726), Bacillus sp. KSM-N440 (FERM P-19728), Bacillus sp. KSM-N659 (FERM P-19730), etc.; cellulase mixtures derived from Trichoderma viride, Aspergillus acleatus, Clostridium thermocellum, Clostridium stercorarium, Clostridium josui, Cellulomonas fimi, Acremonium celluloriticus, Irpex lacteus, Aspergillus niger, and Humicola insolens; and heat-resistant cellulase derived from Pyrococcus horikoshii. Of these, cellulase derived from Trichoderma reesei, Trichoderma viride, or Humicola insolens is preferably employed. For example, Celluclast 1.5L (trade name, product of Novozymes), TP-60 (trade name, product of Meiji Co., Ltd.), CellicCTec2 (trade name, product of Novozymes), Accellerase DUET (trade name, product of Genencor), or Ultraflo L (trade name, product of Novozymes) is preferably used.

Specific examples of the β-glucosidase, which is a type of cellulase, include enzymes derived from Aspergillus niger (e.g., Novozyme 188 (trade name, product of Novozymes) or β-glucosidase manufactured by Megazyme), β-glucosidase derived from Trichoderma reesei, and β-glucosidase derived from Penicillium emersonii.

Specific examples of the hemicellulase include hemicellulase products derived from Trichoderma reesei such as CellicHTec2 (trade name, product of Novozymes); xylanase derived from Bacillus sp. KSM-N546 (FERM P-19729); xylanase derived from Aspergillus niger, Trichoderma viride, Humicola insolens, or Bacillus alcalophilus; and xylanase derived from the genus Thermomyces, Aureobasidium, Streptomyces, Clostridium, Thermotoga, Thermoascus, Caldocellum, or Thermomonospora.

The enzyme employed in step (2) is preferably one or more species selected from among the aforementioned cellulases and hemicellulases, for improvement of saccharification efficiency.

Conditions for the saccharification treatment of the cellulose raw material with an enzyme in step (2) may be appropriately determined in consideration of the crystallinity of cellulose contained in the cellulose raw material or the type of the enzyme employed.

In the case where, for example, the cellulose raw material derived from bagasse (residue of squeezed sugarcane) is employed as a substrate, the enzyme may added to a 0.5 to 40% (w/v) suspension of the substrate so that the amount of enzyme protein is adjusted to 0.04 to 600 mass % with respect to the substrate, and the enzymatic reaction may be carried out in a buffer having a pH of 2 to 10 under the following conditions: reaction temperature: 10 to 90° C., and reaction time: 30 minutes to five days, preferably 0.5 to three days, to thereby produce a saccharide.

Preferably, the pH of the aforementioned buffer is appropriately determined in consideration of the type of the enzyme employed. The pH is preferably 3 to 7, more preferably 4 to 6.

Also, preferably, the aforementioned reaction temperature is appropriately determined in consideration of the type of the enzyme employed. The reaction temperature is preferably 20 to 70° C., more preferably 40 to 60° C.

In addition to the aforementioned embodiments, the present invention discloses the following production processes.

<1> A method for producing saccharide, including the following steps (1) and (2).

step (1): a step of heating a cellulose raw material in the presence of one or more additives selected from the group consisting of a nonionic surfactant and polyethylene glycol; and water, at a pH lower than 7, preferably a pH of 4 or lower, more preferably 3.0 or lower, even more preferably 2.7 or lower, further more preferably 2.5 or lower, and preferably 0.1 or higher, more preferably 0.5 or higher, even more preferably 1.0 or higher; or preferably 4 or lower, more preferably 0.1 to 3.0, even more preferably 0.5 to 2.7, further more preferably 1.0 to 2.5, to thereby prepare a heat treatment product

step (2): a step of subjecting the treatment product prepared in step (1) to a saccharification treatment by use of an enzyme

<2> The method for producing saccharide as described in <1> above, which method further includes, before step (2), a step of neutralizing the treatment product prepared in step (1) with a base, preferably one or more species selected from among sodium hydroxide, potassium hydroxide, and calcium hydroxide, more preferably sodium hydroxide or calcium hydroxide.
<3> The method for producing saccharide as described in <1> or <2> above, wherein the nonionic surfactant has an HLB value of 3 or higher and 20 or lower, preferably 5 or higher and 20 or lower, or 3 to 20, preferably 5 to 20.
<4> The method for producing saccharide as described in any of <1> to <3> above, wherein the nonionic surfactant has an HLB value of 8 or higher and 20 or lower, preferably 10 or higher and 20 or lower, or 8 to 20, preferably 10 to 20.
<5> The method for producing saccharide as described in any of <1> to <4> above, wherein the nonionic surfactant has an HLB value of 12 or higher and 20 or lower, preferably 16 or higher and 20 or lower, or 12 to 20, preferably 16 to 20.
<6> The method for producing saccharide as described in any of <1> to <5> above, wherein the nonionic surfactant is one or more species selected from the group consisting of polyoxyethylene alkyl ethers, polyoxyethylene sorbitan fatty acid esters, polyoxyethylene sorbitol fatty acid esters, and polyoxyethylene fatty acid esters, preferably one or more species selected from the group consisting of polyoxyethylene sorbitan laurate, polyoxyethylene sorbitan stearate, polyoxyethylene lauryl ether, polyethylene glycol monolaurate, and polyethylene glycol monostearate.
<7> The method for producing saccharide as described in <6> above, wherein the nonionic surfactant is one or more species selected from the group consisting of polyoxyethylene sorbitan fatty acid esters, preferably one or more species selected from the group consisting of polyoxyethylene sorbitan laurate and polyoxyethylene sorbitan stearate.
<8> The method for producing saccharide as described in any of <1> to <7> above, wherein polyethylene glycol has a weight average molecular weight of 200 or higher, preferably 300 or higher, more preferably 400 or higher, and 20,000 or lower, preferably 18,000 or lower, more preferably 15,000 or lower, or 200 to 20,000, preferably 300 to 18,000, more preferably 400 to 15,000.
<9> The method for producing saccharide as described in any of <1> to <8> above, wherein polyethylene glycol has a weight average molecular weight of 1,000 or higher, preferably 1,500 or higher, and 10,000 or lower, preferably 6,000 or lower, or 1,000 to 10,000, preferably 1,500 to 6,000.
<10> The method for producing saccharide as described in any of <1> to <9> above, wherein the total amount of the additives used in step (1) is 0.1 mass % or more, with respect to the dry weight of the cellulose raw material, preferably 0.2 mass % or more, more preferably 1 mass % or more, and 80 mass % or less, preferably 50 mass % or less, more preferably 20 mass % or less, or 0.1 to 100 mass %, preferably 0.2 to 80 mass %, more preferably 0.2 to 50 mass %, even more preferably 1 to 20 mass %.
<11> The method for producing saccharide as described in any of <1> to <10> above, wherein the heating treatment of step (1) is performed at 100° C. or higher, preferably 120° C. or higher, more preferably 140° C. or higher, 300° C. or lower, and preferably 250° C. or lower, more preferably 230° C. or lower, or 100 to 300° C., preferably 120 to 250° C., more preferably 140 to 230° C.
<12> The method for producing saccharide as described in any of <1> to <11> above, wherein the heating treatment of step (1) is performed at a pressure equal to or higher than a saturated steam pressure, preferably 0.1 MPa or higher, more preferably 0.2 MPa or higher, even more preferably 0.3 MPa or higher, further more preferably 0.5 MPa or higher, and 10 MPa or lower, preferably 8 MPa or lower, more preferably 6 MPa or lower, or preferably 0.1 to 10 MPa, more preferably 0.2 to 8 MPa, even more preferably 0.3 to 6 MPa, further more preferably 0.5 to 6 MPa.
<13> The method for producing saccharide as described in any of <1> to <12> above, wherein an aqueous slurry having a cellulose raw material roughly milled product content of 1 g/L or more, preferably 5 g/L or more, more preferably 8 g/L or more, and 500 g/L or less, preferably 400 g/L or less, more preferably 300 g/L or less, or 1 to 500 g/L, preferably 5 to 400 g/L, more preferably 8 to 300 g/L is subjected to the heating treatment of step (1) in the presence of said additives.
<14> The method for producing saccharide as described in any of <1> to <13> above, wherein the enzyme used in step (2) is one or more species selected from cellulase and hemicellulase.
<15> The method for producing saccharide as described in any of <1> to <14> above, wherein, in step (2), the cellulose raw material is subjected to the saccharification treatment in a buffer having a pH of 2 or higher, preferably 3 or higher, more preferably 4 or higher, and a pH of 10 or lower, preferably 7 or lower, more preferably 6 or lower, or a pH of 2 to 10, preferably 3 to 7, more preferably 4 to 6.
<16> The method for producing saccharide as described in any of <1> to <15> above, wherein, in step (2), the cellulose raw material is subjected to a saccharification treatment by use of an enzyme at a reaction temperature of 10° C. or higher, preferably 20° C. or higher, more preferably 40° C. or higher, and 90° C. or lower, preferably 70° C. or lower, more preferably 60° C. or lower, or 10 to 90° C., preferably 20 to 70° C., more preferably 40 to 60° C.

### EXAMPLES

In the below-described Examples, unless otherwise specified, the symbol “%” refers to “mass %,” exclusive of crystallinity (%). The cellulose content of a cellulose raw material corresponds to the holocellulose content thereof.

### (1) Calculation of Holocellulose Content of Cellulose Raw Material

A milled cellulose raw material was subjected to Soxhlet extraction with an ethanol-dichloroethane solvent mixture (1:1) for six hours, and the extracted sample was dried under vacuum at 60° C. To 2.5 g of the resultant sample were added 150 mL of water, 1.0 g of sodium chlorite, and 0.2 mL of acetic acid, and the mixture was heated at 70 to 80° C. for one hour. Subsequently, addition of sodium chlorite and acetic acid and heating were repeatedly carried out three to four times until the color of the sample was removed; i.e., the sample became white. The resultant white residue was subjected to filtration with a glass filter (1G-3), followed by washing with cold water and acetone. The resultant product was dried at 105° C. until the weight thereof reached a constant value, and then the weight of the residue was measured. The holocellulose content was calculated on the basis of the following formula, and the thus-calculated value was regarded as cellulose content.

Cellulose content (mass %)=[the weight of residue (g)/the amount of collected cellulose-containing raw material (g: as reduced to dry raw material, exclusive of basic compound)]×100

### (2) Calculation of Amount by Mole of Anhydroglucose Unit (AGU)

The amount by mole of AGU was calculated on the basis of the following formula under the assumption that all the holoceluloses contained in the cellulose-containing raw material were cellulose.

Amount by mole of AGU=weight of holocellulose (g)/162

### (3) Measurement of Water Content of Cellulose Raw Material

The water content of a cellulose raw material was measured at 150° C. by means of an infrared moisture meter (product name “FD-610,” product of Kett Electric Laboratory). The point at which the rate of change in weight for 30 seconds was 0.1% or less was regarded as the end point of measurement. The thus-measured water content was converted to mass % with respect to the dry weight of the cellulose-containing raw material.

### (4) Determination of Percent Saccharification

In Examples and Comparative Examples, quantitative determination of a saccharide was carried out through the DNS method (“Experimental Biochemistry, Determination of reducing sugar” Gakkai Shuppan Center).

After completion of the saccharification treatment in step (2), the residue and the supernatant were separated from each other through centrifugation. An appropriate amount of the supernatant was added to 1 mL of a DNS solution (0.5% 3,5-dinitrosalicylic acid, 30% sodium potassium tartrate tetrahydrate, and 1.6% sodium hydroxide), and the mixture was heated at 100° C. for five minutes for color development. After cooling, the mixture was analyzed through colorimetry at a wavelength of 535 nm. The amount of reducing saccharide contained in the supernatant was determined by a calibration curve prepared by use of glucose as a standard saccharide.

On the basis of the thus-determined amount of reducing saccharide, percent saccharification was determined. Percent saccharification was calculated by use of the following formula.

Percent saccharification (%)=the reducing saccharide concentration of the supernatant (g/mL)/(cellulose-containing milled product concentration (g/mL (as reduced to dry raw material, exclusive of basic compound))×holocellulose content (g/g−cellulose-containing raw material)/0.9 (molecular weight of glucose/molecular weight of AGU))

### Example 1

### Rough Milling Treatment of Cellulose Raw Material

Bagasse [residue of squeezed sugarcane, holocellulose content: 71.3 mass %, crystallinity: 29%, and water content: 7.0 massa] was used as a cellulose raw material. The cellulose raw material was fed to a batch-type vibration mill (trade name “MB-1,” product of Chuo Kakohki Co., Ltd., total container volume: 3.5 L, rods: 13 SUS304 rods each having φ 30 mm, a length of 218 mm, and a circular cross section, percent filling of rods: 57%). The milling treatment was carried out for 5 minutes, to thereby prepare a roughly milled product.

### (Step (1))

To a glass-made special reactor, there were added 150 mg (dry weight) of the rough milling product, 65 mg of 1M sulfuric acid, 15 mg (effective amount: 15 mg, corresponding to 10 mass % to the weight of dry bagasse) of a nonionic surfactant as an additive; i.e., polyoxyethylene sorbitan laurate (product of Kao Corporation, trade name “Rheodol TW-L120,” HLB: 16.7), and 2,388 mg (such an amount that the cellulose raw material content was adjusted to about 6 mass %) of ion-exchange water. The mixture was sufficiently stirred, and then heated by means of a microwave heater (product of Biotage, trade name “initiator sixty”) at 1.0 MPa and 180° C. for 2 minutes, to thereby prepare a treatment product. The pH during the heating treatment was 1.8.

### (Neutralization Step)

The thus-prepared treatment product was cooled to room temperature, and then 130 mg of 1M aqueous sodium hydroxide was added to the reactor containing the treatment product, to thereby neutralize the product. Subsequently, 0.3 mL of a 100 mM acetate buffer was added thereto, to thereby adjust the volume to 3 mL having a pH of 5.0.

### (Step (2))

Cellulase enzyme preparation CellicCTec2 (trade name, product of Novozymes) was added in such an amount that the enzyme protein amount, with respect to the thus-neutralized treatment product (amount: corresponding to 150 mg as reduced to dry raw material), was adjusted to 1.5 mg. The mixture was subjected to a saccharification treatment at 50° C. for 24 hours under shaking/stirring.

After completion of reaction, the residue and the supernatant were separated from each other through centrifugation. The amount of reducing saccharide released in the supernatant was determined through the aforementioned DNS method, to thereby determine percent saccharification. Table 1 shows the results.

### Example 2

The procedure of Example 1 was repeated, except that 15 mg (effective amount: 15 mg, corresponding to 10 mass % to the weight of dry bagasse) of a nonionic surfactant, polyoxyethylene sorbitan stearate (product of Kao Corporation, trade name “Rheodol TW-S120,” HLB: 14.9), was used as an additive, to thereby produce a saccharide. Table 1 shows the results.

### Example 3

The procedure of Example 1 was repeated, except that 15 mg (effective amount: 15 mg, corresponding to 10 mass % to the weight of dry bagasse) of a nonionic surfactant, sorbitan monostearate (product of Kao Corporation, trade name “Rheodol SP-S10V,” HLB: 4.7), was used as an additive, to thereby produce a saccharide. Table 1 shows the results.

### Example 4

The procedure of Example 1 was repeated, except that 15 mg (effective amount: 15 mg, corresponding to 10 mass % to the weight of dry bagasse) of polyethylene glycol (product of ALDRICH, average molecular weight: 4,400), was used as an additive, to thereby produce a saccharide. Table 1 shows the results.

### Example 5

The procedure of Example 1 was repeated, except that 15 mg (effective amount: 15 mg, corresponding to 10 mass % to the weight of dry bagasse) of polyethylene glycol (product of MP Biomedicals, average molecular weight: 8,000), was used as an additive, to thereby produce a saccharide. Table 1 shows the results.

### Example 6

The procedure of Example 1 was repeated, except that 15 mg (effective amount: 15 mg, corresponding to 10 mass % to the weight of dry bagasse) of a nonionic surfactant, polyoxyethylene lauryl ether (product of Kao Corporation, trade name “Emulgen 120,” HLB: 15.3), was used as an additive, to thereby produce a saccharide. Table 1 shows the results.

### Example 7

The procedure of Example 1 was repeated, except that 15 mg (effective amount: 15 mg, corresponding to 10 mass % to the weight of dry bagasse) of a nonionic surfactant, polyethylene glycol monolaurate (product of Kao Corporation, trade name “Emanon 1112,” HLB: 13.7), was used as an additive, to thereby produce a saccharide. Table 1 shows the results.

### Example 8

The procedure of Example 1 was repeated, except that 15 mg (effective amount: 15 mg, corresponding to 10 mass % to the weight of dry bagasse) of a nonionic surfactant, polyethylene glycol monostearate (product of Kao Corporation, trade name “Emanon 3199V,” HLB: 19.4), was used as an additive, to thereby produce a saccharide. Table 1 shows the results.

### Example 9

The procedure of Example 1 was repeated, except that 15 mg (effective amount: 15 mg, corresponding to 10 mass % to the weight of dry bagasse) of a nonionic surfactant, sorbitan monolaurate (product of Kao Corporation, trade name “Rheodol SB-L10,” HLB: 8.6), was used as an additive, to thereby produce a saccharide. Table 1 shows the results.

### Example 10

The procedure of Example 1 was repeated, except that 15 mg (effective amount: 15 mg, corresponding to 10 mass % to the weight of dry bagasse) of a nonionic surfactant, polyoxyethylene sorbitol fatty acid ester (product of Kao Corporation, trade name “Rheodol 460V,” HLB: 13.8), was used as an additive, to thereby produce a saccharide. Table 1 shows the results.

### Comparative Example 1

The procedure of Example 1 was repeated, except that 15 mg (effective amount: 15 mg, corresponding to 10 mass % to the weight of dry bagasse) of a nonionic surfactant, sorbitan monostearate (product of Kao Corporation, trade name “Rheodol SP-S10V,” HLB: 4.7), was used as an additive, that the nonionic surfactant was added in step (2) instead of step (1), and that the amount of ion-exchange water on step (1) was changed to a value shown in Table 1, to thereby produce a saccharide. Table 1 shows the results.

### Comparative Example 2

The procedure of Example 1 was repeated, except that 15 mg (effective amount: 15 mg, corresponding to 10 mass % to the weight of dry bagasse) of polyethylene glycol (product of MP Biomedicals, average molecular weight: 8,000), was used as an additive, that the additive was added in step (2) instead of step (1), and that the amount of ion-exchange water on step (1) was changed to a value shown in Table 1, to thereby produce a saccharide. Table 1 shows the results.

### Comparative Example 3

The procedure of Example 1 was repeated, except that step (1) was performed without use of an additive, and that the amount of ion-exchange water on step (1) was changed to a value shown in Table 1, to thereby produce a saccharide. Table 1 shows the results.

TABLE 1

| | | | | Ex. 1 | Ex. 2 | Ex. 3 | Ex. 4 | Ex. 5 | Ex. 6 | Ex. 7 |
|---|---|---|---|---|---|---|---|---|---|---|
| Step (1) | Cellulose raw material | | Dry bagasse | 150 | 150 | 150 | 150 | 150 | 150 | 150 |
| | (mg) | | | | | | | | | |
| | Additive | Nonionic | POE sorbitan laurate* 1 | 15 | — | — | — | — | — | — |
| | (mg) | surfactant | POE sorbitan stearate*2 | — | 15 | — | — | — | — | — |
| | | | Sorbitan monostearate*3 | — | — | 15 | — | — | — | — |
| | | | POE lauryl ether*4 | — | — | — | — | — | 15 | — |
| | | | PEG monolaurate*5 | — | — | — | — | — | — | 15 |
| | | | PEG monostearate*6 | — | — | — | — | — | — | — |
| | | | Sorbitan monolaurate*7 | — | — | — | — | — | — | — |
| | | | POEsorbitol fatty acid | — | — | — | — | — | — | — |
| | | | ester*8 | | | | | | | |
| | | PEG | Mw 4400 | — | — | — | 15 | — | — | — |
| | | | Mw 8000 | — | — | — | — | 15 | — | — |
| | Water (mg) | | | 2388 | 2388 | 2388 | 2388 | 2388 | 2388 | 2388 |
| | Reaction | | Concentration of cellulose | 6 | 6 | 6 | 6 | 6 | 6 | 6 |
| | conditions | | raw material (mass %) | | | | | | | |
| | | | Amount of additive tocell- | 10 | 10 | 10 | 10 | 10 | 10 | 10 |
| | | | ulose raw material (mass %) | | | | | | | |
| | | | pH | 1.8 | 1.8 | 1.8 | 1.8 | 1.8 | 1.8 | 1.8 |
| | | | Max. temp. (° C.) | 180 | 180 | 180 | 180 | 180 | 180 | 180 |
| | | | Retention time (min) | 2 | 2 | 2 | 2 | 2 | 2 | 2 |
| Neutralization | Neutralizing agent | | | NaOH | NaOH | NaOH | NaOH | NaOH | NaOH | NaOH |
| step | | | | | | | | | | |
| Step (2) | Additive | Nonionic | Sorbitan monostearate*3 | — | — | — | — | — | — | — |
| | (mg) | surfactant | | | | | | | | |
| | | PEG | Mw 8000 | — | — | — | — | — | — | — |
| | Treatment conditions | | Enzyme | CTec2 | CTec2 | CTec2 | CTec2 | CTec2 | CTec2 | CTec2 |
| | | | | *9 | *9 | *9 | *9 | *9 | *9 | *9 |
| | | | Time (hr) | 24 | 24 | 24 | 24 | 24 | 24 | 24 |

| | | | | | | | | |
|---|---|---|---|---|---|---|---|---|
| Evaluation | Percent saccharification (%) | 100.0 | 93.5 | 77.0 | 100.0 | 81.4 | 91.0 | 95.5 |

| | | | | | | | Comp. | Comp. | Comp. |
|---|---|---|---|---|---|---|---|---|---|
| | | | | Ex. 8 | Ex. 9 | Ex. 10 | Ex. 1 | Ex. 2 | Ex. 3 |
| Step (1) | Cellulose raw material | | Dry bagasse | 150 | 150 | 150 | 150 | 150 | 150 |
| | (mg) | | | | | | | | |
| | Additive | Nonionic | POE sorbitan laurate*1 | — | — | — | — | — | — |
| | (mg) | surfactant | POE sorbitan stearate*2 | — | — | — | — | — | — |
| | | | Sorbitan monostearate*3 | — | — | — | — | — | — |
| | | | POE lauryl ether*4 | — | — | — | — | — | — |
| | | | PEG monolaurate*5 | — | — | — | — | — | — |
| | | | PEG monostearate*6 | 15 | — | — | — | — | — |
| | | | Sorbitan monolaurate*7 | — | 15 | — | — | — | — |
| | | | POEsorbitol fatty acid ester*8 | — | — | 15 | — | — | — |
| | | PEG | Mw 4400 | — | — | — | — | — | — |
| | | | Mw 8000 | — | — | — | — | — | — |
| | Water (mg) | | | 2388 | 2388 | 2388 | 2403 | 2403 | 2403 |
| | Reaction | | Concentration of cellulose raw | 6 | 6 | 6 | 6 | 6 | 6 |
| | conditions | | material (mass %) | | | | | | |
| | | | Amount of additive to cellulose | 10 | 10 | 10 | — | — | — |
| | | | raw material (mass %) | | | | | | |
| | | | pH | 1.8 | 1.8 | 1.8 | 1.8 | 1.8 | 1.8 |
| | | | Max. temp. (° C.) | 180 | 180 | 180 | 180 | 180 | 180 |
| | | | Retention time (min) | 2 | 2 | 2 | 2 | 2 | 2 |
| Neutralization | Neutralizing agent | | | NaOH | NaOH | NaOH | NaOH | NaOH | NaOH |
| step | | | | | | | | | |
| Step (2) | Additive | Nonionic | Sorbitan monostearate*3 | — | — | — | 15 | — | — |
| | (mg) | surfactant | | | | | | | |
| | | PEG | Mw 8000 | — | — | — | — | 15 | — |
| | Treatment conditions | | Enzyme | CTec2 | CTec2 | CTec2 | CTec2 | CTec2 | CTec2 |
| | | | | *9 | *9 | *9 | *9 | *9 | *9 |
| | | | Time (hr) | 24 | 24 | 24 | 24 | 24 | 24 |

| | | | | | | | |
|---|---|---|---|---|---|---|---|
| Evaluation | Percent saccharification (%) | 91.4 | 81.4 | 84.8 | 72.4 | 75.7 | 70.6 |

*1: Rheodol TW-L120, polyoxyethylene sorbitan laurate (product of Kao Corporation, HLB: 16.7)
*2: Rheodol TW-S120, polyoxyethylene sorbitan stearate (product of Kao Corporation, HLB: 14.9)
*3: Rheodol SP-S10V, sorbitan monostearate (product of Kao Corporation, HLB: 4.7)
*4: Emulgen 120, polyoxyethylene lauryl ether (product of Kao Corporation, HLB: 15.3)
*5: Emanon 1112, polyethylene glycol monolaurate (product of Kao Corporation, HLB: 13.7)
*6: Emanon 3199V, polyethylene glycol monostearate (product of Kao Corporation, HLB: 19.4)
*7: Rheodol SP-L10, sorbitan monolaurate (product of Kao Corporation, HLB: 8.6)
*8: Rheodol 460V, polyoxyethylene sorbitol fatty acid ester (product of Kao Corporation, HLB: 13.8)
*9: CTec2: cellulase enzyme preparation CellicCTec2 (trade name, product of Novozymes)

As is clear from Table 1, the method for producing saccharide according to the present invention provided a more improved percent saccharification, as compared with Comparative Example 3, in which no additive was added in step (1) or step (2), and with Comparative Examples 1 and 2, in which no additive was used in step (1) and the same additive as employed in Examples 3 and 5 was used in step (2).

### INDUSTRIAL APPLICABILITY

The method for producing saccharide according to the present invention attains high saccharide productivity and enables effective production of saccharide from a cellulose raw material. The thus-produced saccharide is useful in, for example, production of ethanol or lactic acid via a fermentation process.

1. A method for producing saccharide, comprising the following steps (1) and (2):
step (1): a step of heating, at 140° C. to 300° C., a cellulose raw material in the presence of one or more additives selected from the group consisting of a nonionic surfactant and polyethylene glycol; and water, at a pH of 0.1 to 3.0, to thereby prepare a heat treatment product;
step (2): a step of subjecting the treatment product prepared in step (1) to a saccharification treatment by use of an enzyme.

2. The method for producing saccharide according to claim 1, which method further comprises, before step (2), a step of neutralizing the treatment product prepared in step (1) with a base.

3. The method for producing saccharide according to claim 1, wherein the nonionic surfactant has an HLB value of 3 to 20.

4. The method for producing saccharide according to claim 1, wherein the nonionic surfactant has an HLB value of 8 to 20.

5. The method for producing saccharide according to claim 1, wherein the nonionic surfactant has an HLB value of 12 to 20.

6. The method for producing saccharide according to claim 1, wherein the nonionic surfactant is one or more species selected from the group consisting of polyoxyethylene alkyl ethers, polyoxyethylene sorbitan fatty acid esters, polyoxyethylene sorbitol fatty acid esters, and polyoxyethylene fatty acid esters.

7. The method for producing saccharide according to claim 6, wherein the nonionic surfactant is a polyoxyethylene sorbitan fatty acid ester.

8. The method for producing saccharide according to claim 1, wherein the polyethylene glycol has a weight average molecular weight of 200 to 20,000.

9. The method for producing saccharide according to claim 1, wherein the polyethylene glycol has a weight average molecular weight of 1,000 to 10,000.

10. The method for producing saccharide according to claim 1, wherein the total amount of the additives used in step (1) is 0.1 to 100 mass %, with respect to the weight of the cellulose raw material in a dry state.

11. The method for producing saccharide according to claim 1, wherein the heating treatment of step (1) is performed at 140° C. to 250° C.

12. The method for producing saccharide according to claim 1, wherein the heating treatment of step (1) is performed at 250° C. or lower.

13. The method for producing saccharide according to claim 1, wherein the heating treatment of step (1) is performed at 230° C. or lower.

14. The method for producing saccharide according to claim 1, wherein the heating treatment of step (1) is performed at 140 to 230° C.

15. The method for producing saccharide according to claim 1, wherein the pH of step (1) is 2.7 or lower.

16. The method for producing saccharide according to claim 1, wherein the pH of step (1) is 2.5 or lower.

17. The method for producing saccharide according to claim 1, wherein the pH of step (1) is 0.5 or higher.

18. The method for producing saccharide according to claim 1, wherein the pH of step (1) is 1.0 or higher.

19. The method for producing saccharide according to claim 1, wherein the pH of step (1) is 0.5 to 2.7.

20. The method for producing saccharide according to claim 1, wherein the pH of step (1) is 1.0 to 2.5.
